(19) United States
(12) Patent Application Publication
(10) Pub. No.: US 2026/0090839 A1
(43) Pub. Date: Apr. 2, 2026

(54) PRE-SURGICAL ANALYSIS SYSTEM AND METHODS OF PERFORMING THE SAME

(71) Applicant: Prem Ramkumar MD, Inc., Seal Beach, CA (US)

(72) Inventor: Prem Narayan Ramkumar, Seal Beach, CA (US)

(73) Assignee: Prem Ramkumar MD, Inc., Seal Beach, CA (US)

(21) Appl. No.: 19/078,037

(22) Filed: Mar. 12, 2025

Related U.S. Application Data
(60) Provisional application No. 63/702,614, filed on Oct. 2, 2024.

(51) Int. Cl. A61B 34/10; G06T 7/00; G06T 7/11; G06T 7/62; G16H 50/70

(52) U.S. Cl. CPC A61B 34/10; G06T 7/0012; G06T 7/11; G06T 7/62; G16H 50/70; G06T 2207/10081; G06T 2207/30008

(57) ABSTRACT

A pre-surgical analytical system including a plurality of images gathered from a CT scan, a classification unit that classifies the identifies landmark images from the plurality of images, a segmentation unit that segments each of the landmark image; an image analysis unit that calculates a plurality of predetermined angles and measurements of anatomical structures in each image where the image analysis unit compares each measurement and angle to predetermined thresholds and determines the number of angles and measurements are outside the predetermined threshold, and the image analysis unit weighs each of the angles and measurements outside the predetermined thresholds and calculates a score indicating a potential outcome of a surgical procedure.

### BACKGROUND OF THE INVENTION

The integration of computed tomography (CT) scans in the preoperative planning workflow for total knee arthroplasty (TKA) since its initial development in the 1970s offers the opportunity to evaluate the anatomy of patients indicated for the operation. Despite the increasing volume and clinical success of TKA, 15 to 30% of patients remain dissatisfied after the operation. This dissatisfaction has been attributed to patient selection, patient expectations, poorly indicated surgery, implant design limitations, and technical execution. Patient anatomy, both at the level of the knee and throughout the extremity, especially at the hip, has not been commonly implicated as a source of dissatisfaction. Many agree that restoring native anatomy and alignment (ie, kinematic alignment) and/or achieving a predefined target (ie, mechanical alignment) is important in optimizing function and decreasing pain. However, the intraoperative execution of TKA is primarily defined by cuts and releases that accommodate the implant rather than the patient's native anatomy [10]. This is understandable given that the selection and sizing of available implants are defined by manufacturing and resource constraints to accommodate “typical” anatomy.

In response to the consistently reported dissatisfaction rate, characterizing preoperative anatomy is a component of the reevaluation of philosophical, clinical, and technical approaches to TKA. While some imaging techniques have been developed to attempt to categorize patients based on anatomy, processing and managing imaging data poses a high manual labor burden, susceptible to inaccuracies, to segment desired objects from each image reconstructions or measurements. Since each anatomical structure may include hundreds of image slices, the process of analyzing each image becomes laborious and unscalable.

Therefore, a need exists for a system that will analyze anatomical structures in the human body and provide insights into the potential success of corrective surgeries for those structures.

### SUMMARY OF THE INVENTION

Systems, methods, features, and advantages of the present invention will be or will become apparent to one with skill in the art upon examination of the following figures and detailed description. It is intended that all such additional systems, methods, features, and advantages be included within this description, be within the scope of the invention, and be protected by the accompanying claims.

One embodiment of the present disclosure includes a pre-surgical analytical system including a plurality of images gathered from a CT scan, a classification unit that classifies the identifies landmark images from the plurality of images, a segmentation unit that segments each of the landmark images, an image analysis unit that calculates a plurality of predetermined angles and measurements of anatomical structures in each image wherein the image analysis unit compares each measurement and angle to predetermined thresholds and determines the number of angles and measurements that are outside the predetermined threshold, and the image analysis unit weighs each of the angles and measurements outside the predetermined thresholds and calculates a score indicating potential phenotypes or classifications of an anatomic structure and/or potential recommendations with associated outcomes of a surgical procedure.

In another embodiment, the images are configured to a 1 mm×1 mm×1 mm voxel prior to analysis.

In another embodiment, each image is a two-dimensional slice of an anatomical structure.

In another embodiment, a deep learning computer model is applied to each image to identify each landmark image.

In another embodiment, the deep learning computer model is a VGG16-XGBoost classification model.

In another embodiment, the anatomical structure is the knee.

In another embodiment, the anatomical structure is the hip.

In another embodiment, the threshold values are based on historical surgical information.

In another embodiment, 27 unique angles and measurements are taken from each image.

In another embodiment, the angles and measurements include the rotational measurements of the anatomical structure.

Another embodiment of the present disclosure includes a method of performing a pre-surgical analysis using a pre-surgical analytical system including the steps of gathering a plurality of images from a CT scan, classifying the identified landmark images from the plurality of images, segmenting each of the landmark image, calculating a plurality of predetermined angles and measurements of anatomical structures in each image, comparing each measurement and angle to predetermined thresholds, determining the number of angles and measurements that are outside the predetermined threshold, and weighing each of the angles and measurements outside the predetermined thresholds and calculating a score indicating a potential outcome of a surgical procedure.

Another embodiment includes the step of configuring each image to a 1 mm×1 mm×1 mm voxel prior to analysis.

In another embodiment, each image is a two-dimensional slice of an anatomical structure.

Another embodiment includes the step of applying a deep learning computer model to each image to identify each landmark image.

In another embodiment, the deep learning computer model is a VGG16-XGBoost classification model.

In another embodiment, the anatomical structure is the knee.

In another embodiment, the anatomical structure is the hip.

In another embodiment, the threshold values are based on historical surgical information.

In another embodiment, 27 unique angles and measurements are taken from each image.

In another embodiment, the angles and measurements include the rotational measurements of the anatomical structure.

### BRIEF DESCRIPTION OF THE DRAWINGS

The accompanying drawings, which are incorporated in and constitute a part of this specification, illustrate an implementation of the present invention and, together with the description, serve to explain the advantages and principles of the invention. In the drawings:

FIG. 1 depicts one embodiment of a pre-surgery analytic system 100 consistent with the present invention;

FIG. 2 depicts one embodiment of a pre-surgery analytic unit;

FIG. 3 depicts one embodiment of a communication device 104/106 consistent with the present invention;

FIG. 4 depicts a schematic representation of a process performed by the pre-surgery analytic unit;

FIG. 5 depicts a schematic representation of the process of processing CT scan images;

FIG. 6 depicts examples of the gathering and landmarking of CT scan images;

FIG. 7 depicts a schematic representation of CT scan images analyzed to identify angles and measurements within each CT scan image; and

FIG. 8 depicts a schematic representation of a process used to determine if a patient is a good candidate for a surgical procedure;

FIG. 9 depicts a schematic representation of a process used to determine the classification or outlier status of a patient's anatomy prior to a surgical procedure;

FIG. 10 depicts a schematic representation of a process to generate a digital twin of an anatomical structure;

FIG. 11 depicts a schematic representation of a process performed by the pre-surgery analytic unit;

FIG. 12 depicts a schematic representation of a process performed by the pre-surgery analytic unit to classify images; and

FIG. 13 depicts a schematic representation of a process performed by the pre-surgery analytic unit to segment images.

### DETAILED DESCRIPTION OF THE INVENTION

Referring now to the drawings which depict different embodiments consistent with the present invention, wherever possible, the same reference numbers will be used throughout the drawings and the following description to refer to the same or like parts.

A pre-surgery analysis system analyzes images collected by a CT scan, scales the images and analyzes each image to classify each image and to determine different structures or anatomic morphologies from the images. The pre-surgery analysis unit calculates and measures a series of variables based on the images and determines if the variables fit within chosen or historical thresholds for the measured variables. A list of variables fitting within chosen or historical thresholds may be analyzed for weighting of each measured variable for a given procedure. In addition, a list of outliers, or variables outside the threshold values, are determined and these outliers are used to generate a patient score based on the weighting of each measured variable for a given procedure. In addition, the anatomical structure can be modeled to calculate potential outcomes based on similar procedures or similar anatomical structures.

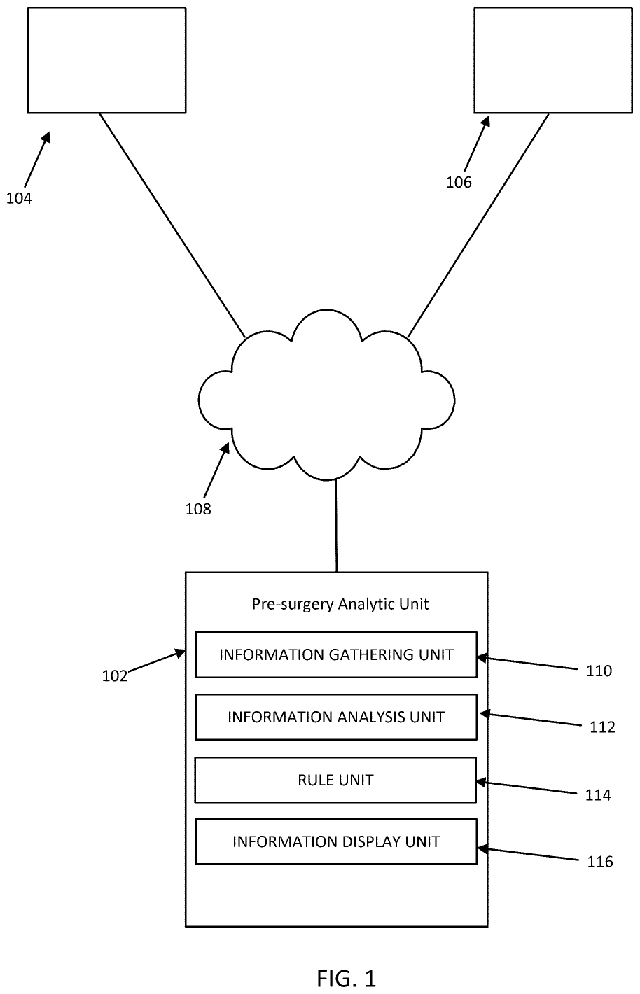

FIG. 1 depicts one embodiment of a pre-surgery analytic system 100 consistent with the present invention. The pre-surgery analytic system 100 includes a pre-surgery analytic unit 102, a communication device 1 (104), a communication device 2 (106), each communicatively connected via a network 108. The pre-surgery analytic unit 102 further includes an information gathering unit 110, an information analysis unit 112, a rule unit 114 and an information display unit 116.

The information gathering unit 110 and information analysis unit 112 may be embodied by one or more servers. Alternatively, each of the rule unit 114 and information display unit 116 may be implemented using any combination of hardware and software, whether as incorporated in a single device or as functionally distributed across multiple platforms and devices.

In one embodiment, network 108 is a cellular network, a TCP/IP network, or any other suitable network topology. In another embodiment, the row identification device may be servers, workstations, network appliances or any other suitable data storage devices. In another embodiment, the communication devices 104 and 106 may be any combination of cellular phones, telephones, personal data assistants, or any other suitable communication devices. In one embodiment, the network 108 may be any private or public communication network known to one skilled in the art such as a local area network (“LAN”), wide area network (“WAN”), peer-to-peer network, cellular network or any suitable network, using standard communication protocols. The network 108 may include hardwired as well as wireless branches. Information gathering unit 112 may include a digital camera.

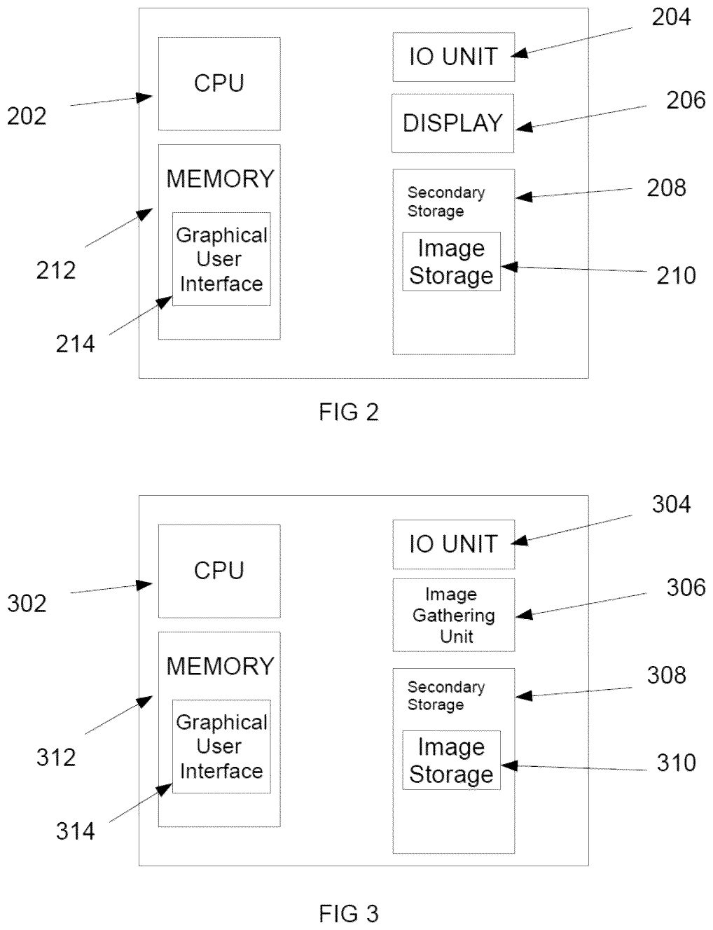

FIG. 2 depicts one embodiment of a pre-surgery analytic unit 102. The pre-surgery analytic unit 102 includes a network I/O device 204, a processor 202, a display 206 and a secondary storage unit 208 running image storage unit 210 and a memory unit 212 running a graphical user interface 214. The image gathering unit 112, operating in memory unit 208 of the pre-surgery analytic unit 102, is operatively configured to receive an image from the network I/O device 204. In one embodiment, processor 202 may be a central processing unit (“CPU”), an application specific integrated circuit (“ASIC”), a microprocessor or any other suitable processing device. Memory unit 212 may include a hard disk, random access memory, cache, removable media drive, mass storage or configuration suitable as storage for data, instructions, and information. In one embodiment, memory 208 and processor 202 may be integrated. The memory unit 208 may use any type of volatile or non-volatile storage techniques and mediums. The network I/O device 204 device may be a network interface card, a cellular interface card, a plain old telephone service (“POTS”) interface card, an ASCII interface card, or any other suitable network interface device. Rule unit 114 may be a compiled program running on a server, a process running on a microprocessor or any other suitable port control software.

FIG. 3 depicts one embodiment of a communication device 104/106 consistent with the present invention. The communication device 104/106 includes a processor 302, a network I/O Unit 304, an image capture unit 306, a secondary storage unit 308 including an image storage device 310, and memory 312 running a graphical user interface 314. In one embodiment, processor 302 may be a central processing unit (“CPU”), an application specific integrated circuit (“ASIC”), a microprocessor or any other suitable processing device. Memory 312 may include a hard disk, random access memory, cache, removable media drive, mass storage or configuration suitable as storage for data, instructions, and information. In one embodiment, memory 312 and processor 302 may be integrated. The memory may use any type of volatile or non-volatile storage techniques and media. The network I/O device 304 may be a network interface card, a plain old telephone service (“POTS”) interface card, an ASCII interface card, or any other suitable network interface device.

In one embodiment, the network 108 may be any private or public communication network known to one skilled in the art such as a Local Area Network (“LAN”), Wide Area Network (“WAN”), Peer-to-Peer Network, Cellular network or any suitable network, using standard communication protocols. The network 108 may include hardwired as well as wireless branches.

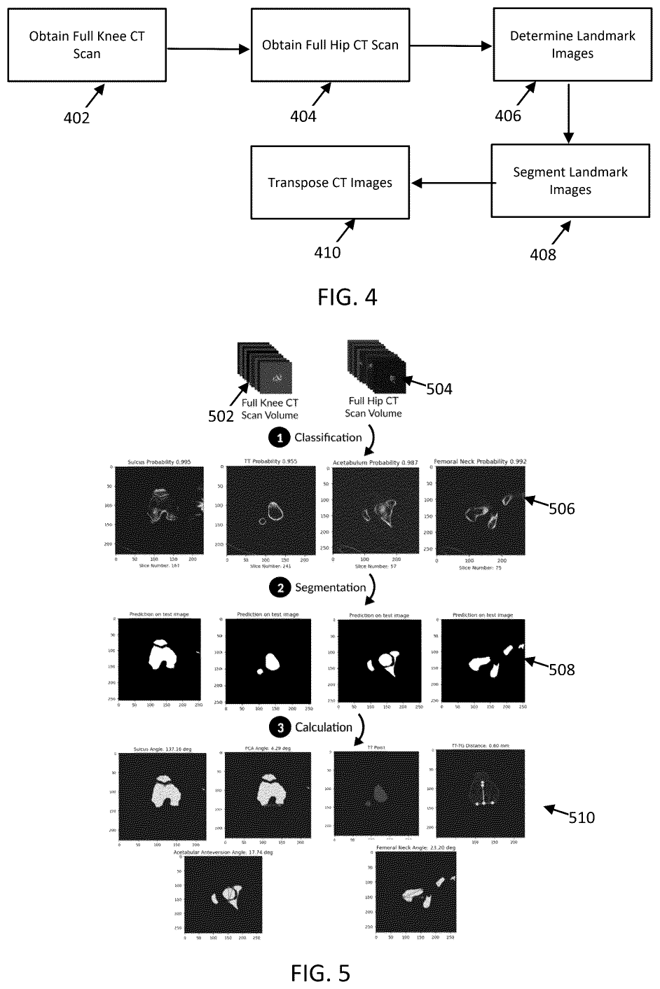

FIG. 4 depicts a schematic representation of a process performed by the pre-surgery analytic unit 102. In step 402 and step 404, a plurality of full knee and full hip CT scan images are gathered. Pixel arrays from the CT scan images are reformatted using pixel spacing metadata to represent the CT scan images as 1.00 mm×1.00 mm×1.00 mm voxels that ensures accuracy in the pixel-to-mm calculations. Volumes in the CT scan images were initially thresholded under the standard Hounsfield range for bones at 300 to 1,400. Location information including X, Y, and Z locations are recorded with an accuracy of 0.01 mm. The distance between CT scan image's positional differences are measured across each CT scan image. In one embodiment, the CT scan images represent two dimensional slices of an anatomical structure.

In step 406, landmark images are determined for each CT scan image. A validated multistep deep learning and computer vision pipeline identifies the highest probability landmark images of the femoral neck, acetabulum, trochlea, and tibial tuberosity from the plurality of hip and knee CT scan images. Deep-convolutional features of each CT scan image are determined using an XGBoost algorithm and a softmax activation function that determines the likelihood of an image being one of a plurality of predetermined classes. Discrete anatomical regions on the hip and knee lower extremities from each landmark CT scan image return the highest probability images for specific landmark classes with accuracy, recall, and precision above 0.87 and 0.90 for hip and knee, respectively. In one embodiment, a user may manually adjust the landmark image by selecting the next higher probability landmark image.

In step 408, each identified landmark image is segmented with a 2-dimensional (2D) TransUNet to create masks with each segmented image being measured using algorithms that rely upon the functionality provided by the OpenCV2 library. Each landmark CT image is segmented (>0.95 mean intersection over union; >0.94 dice coefficient) and made into contours using a computer vision library. From the contours, preliminary measurements are obtained from landmark points in each landmark CT scan image, which are adjusted or corrected quickly by users through an interactive 2D image by clicking at specific pixels in the landmark CT image. Each identified point is translated into a patient-coordinate system by calculating the offset of the clicked pixel to the landmark CT image's metadata. In step 410, each landmark CT image is transposed to represent images in coronal and sagittal views.

FIG. 5 depicts a schematic representation of the process of processing CT scan images. CT full knee scans 502 and CT full hip scans 504. Each CT scan image is classified based on a probability determined by analyzing each CT scan image using a deep learning model. In one embodiment, the deep learning model is a VGG16-XGBoost classification model. The classified images 506 are segmented into landmark images using a Unet convolution. In one embodiment, the Unet convolution is a TransUNet convolution. Each segmented image is analyzed to measure each object in the segmented image with masks being applied to each identified structure in the image.

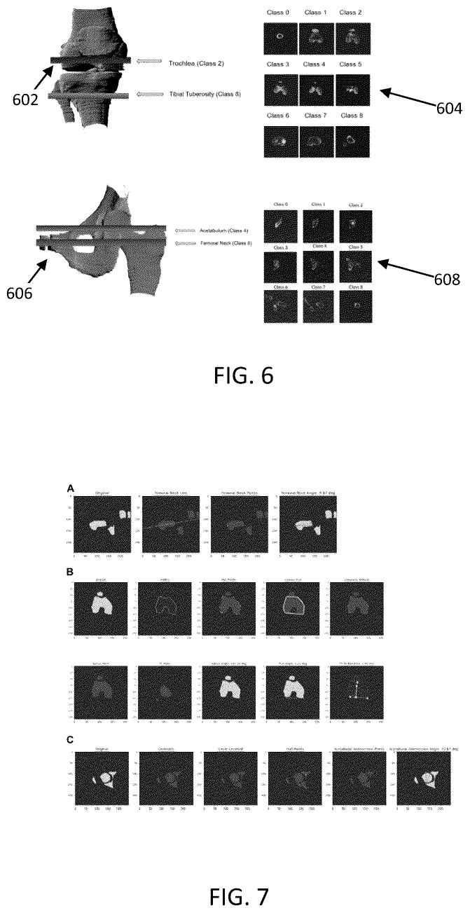

FIG. 6 depicts examples of the gathering of CT scan images. For the knee 602, slices of different areas of the knee are extracted from a CT scan representing different structures in the knee and are stored as CT scan knee images 604. Similarly, for the hip 606, slices of different areas of the hip are extracted from a CT scan representing different structures in the hip and are stored as CT scan hip images 608.

FIG. 7 depicts a schematic representation of CT scan images analyzed to identify angles and measurements within each CT scan image. Using the coronal view of each CT scan image, three (3) angular and two (2) distance measurements are calculated in each CT scan image. The neck-shaft angle is measured taking bisecting axis of the neck and calculating the angular difference between the axes defined by the femoral canal. The hip-knee-ankle axis (HKAA) is measured by first projecting the center of the femoral head, the center of the distal femur, the center of the tibial spine, and the center of the ankle into a 3D patient coordinate system. The intersection point between the hip-distal femur axis and the tibial spine-ankle axis is calculated and the HKAA angle is identified as the angle between the center of the hip, the intersection point, and the center of the ankle.

Using the calculations and CT scan images, individuals are categorized as varus if the intersection point was lateral to the hip/ankle axis and are conversely categorized as valgus if the intersection point was medial to the hip-ankle axis. The tibiofemoral angle (TFA) is calculated by measuring the angle formed between the axis defined by the femoral canal and the axis defined by the tibial canal. The condylar width is measured from the axis of the theoretical distal resection line. Lateral and medial tibial widths are measured from the peak of the tibial spine to the most lateral or most medial points of the plateau along the resection line.

In the axial view, hip and knee rotational measurements are determined using the CT scan images. In knee 602, the posterior condylar axis (PCA) angle is determined by defining the most posterior point axis angle with respect to the x-axis. The transepicondylar axis (TEA) is determined using the anatomic axis connecting the eminences of the medial and lateral femoral condyles. Rotational measurements take into account the x-axis (or the floor) as well as with respect to the PCA (twist angle). The sulcus angle is determined by analyzing the deepest point of the trochlear groove and the most extreme convex hull points of the trochlea. The tibial tuberosity-trochlear groove (TT-TG) distance is determined by taking the most anterior point of the TT and the deepest point of the trochlea, projecting them perpendicular to the PCA, and calculating the Euclidean distance.

In the hip, the femoral neck angle is determined by bisecting the neck axis and calculating the angle relative to the x-axis. Femoral torsion is determined by taking the absolute value of the difference between the angle between the femoral neck axis and the PCA. The relative femoral version is determined by projecting the PCA to the x-axis and then adding or subtracting the PCA angle from the femoral neck angle leading to values that can be both negative (femoral retroversion) and positive (femoral anteversion). The acetabular version is determined by identifying the axis of the medial wall and calculating the angle of the perpendicular bisector with respect to the x-axis. The width of the femoral head is determined by measuring the distance of the diameter.

In the sagittal view, the medial and lateral posterior tibial slope (PTS) are determined by analyzing the canal angle with respect to the axis of the most anterior and posterior points on the lateral and medial plateaus. The anteroposterior (AP) tibial distance is determined from the most anterior point on the tibial plateau to the most posterior point on the tibial plateau. Normative distribution plots for each of the 27 linear and angular metrics previously discussed. The thresholds for the 15th percentile at both extremes are calculated and a variable that did not fall within the central 70th percentile distribution was considered an outlier index.

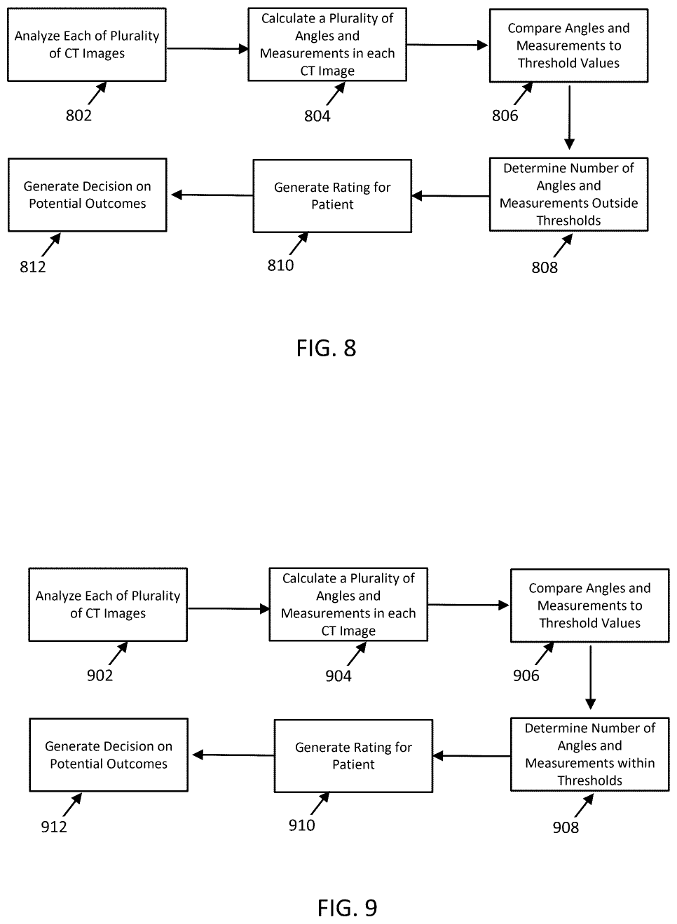

FIG. 8 depicts a schematic representation of a process used to determine if a patient is a good candidate for a surgical procedure. In step 802, a plurality of CT scan images of a patient is collected and analyzed to determine various structures in the images using the methods previously described. In step 804, a plurality of angles and measurements are calculated using the analyzed images including the angles and measurements previously discussed. In step 806, each of the angles and measurements is compared to a threshold range to determine whether the value is inside or outside of the range. In one embodiment, the threshold ranges for each angle or measurement are determined by an analysis of historic values or by a comparison amongst each other. In step 808, the number of angles and measurements outside the threshold range or as morphometric cluster is determined. In step 810, a rating for the patient is generated by applying weighting metrics to the angles and measurements that are outside the threshold range with the weighting metrics being applied based on the type of measurement and angle measured and the effect of the measurement or angle on the historical outcomes of prior procedures. In step 812, a decision on potential outcomes for the patient are determined based on the rating of the patient.

FIG. 9 depicts a schematic representation of a process used to determine the classification or outlier status of a patient's anatomy prior to a surgical procedure. In step 902, a plurality of CT scan images of a patient is collected and analyzed to determine various structures in the images using the methods previously described. In step 904, a plurality of angles and measurements are calculated using the analyzed images including the angles and measurements previously discussed. In step 906, each of the angles and measurements is compared to a threshold range to determine whether the value is inside or outside of the range. In one embodiment, the threshold ranges for each angle or measurement are determined by an analysis of historic values or by a comparison amongst each other. In step 908, each of the angles and measurements are compared to the threshold range or as morphometric cluster is determined. In step 910, a rating for the patient is generated by applying weighting metrics to the angles and measurements based on the numerical deviations from threshold values with the weighting metrics being applied based on the type of measurement and angle measured and the effect of the measurement or angle on the historical outcomes of prior procedures. This rating may be whether the anatomy is an outlier or a morphometric classification. In step 912, the rating would be used in the preoperative or intraoperative state to generate suggestions, recommendations, or insights that could affect a surgical outcome.

As an illustrative example of the system results being utilized in a surgical procedure preparation. The pre-surgical analytic system 100 can identify 3D patient morphology to preoperatively plan a generalized approach such as mechanical alignment, kinematic alignment, implant sizing, and implant position. As another illustrative example, the pre-surgical analytic system 100 may identify 3D patient morphology to determine intraoperative technical execution of a reconstructive knee surgery including resection depth and angular resection.

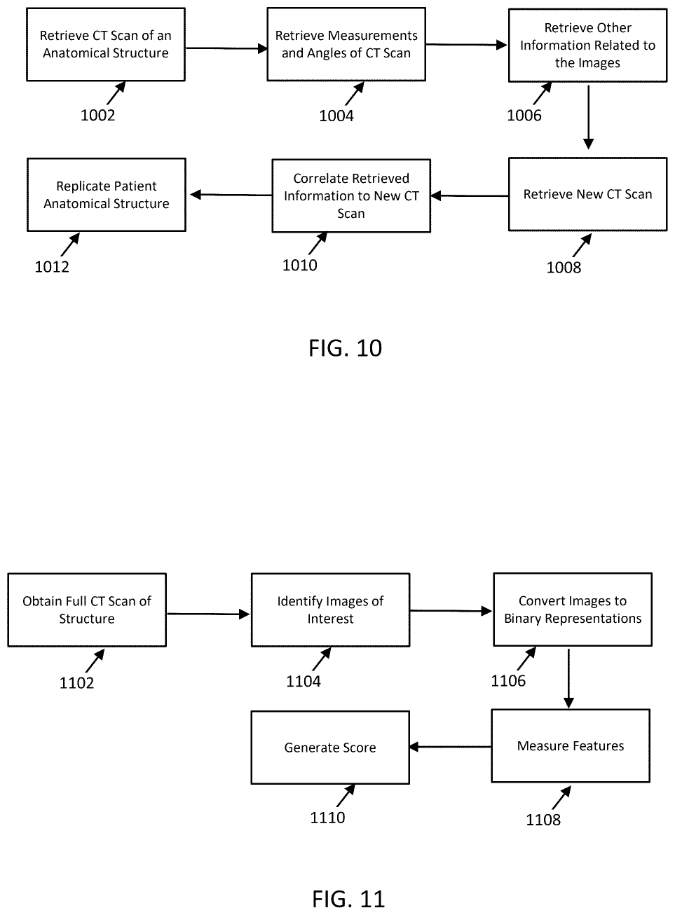

FIG. 10 depicts a schematic representation of a process to generate a digital twin of an anatomical structure. In step 1002, CT scans of anatomical structures are gathered from a group of historical CT scans. Each CT Scan includes a plurality of measurements and angles determined from the anatomical structure with the measurements and angles measured being determined based on the anatomical structure targeted for analysis. In step 1004, the measurements and angles for the gathered CT scans having the same or similar anatomical structure are retrieved. In step 1006, other information related to the anatomical structure that is targeted in the CT scans are retrieved. The other information may include, but is not limited to, patient demographic information, patient age, patient medical history, patient outcome and any other information related to the patient or anatomical structure. In step 1008, a new CT scan of the anatomical structure in the patient targeted for surgery. In one embodiment, the new CT scan is a stream of images from a video capture device. In step 1010, each piece of retrieved information is related to other pieces of information for the patient targeted for surgery by applying rules to the information that result in each piece of information being logically related to at least one other piece of information. In one embodiment, the rules are specific to the target anatomical structure. In another embodiment, the rules are related to similar anatomical structures. In step 1012, retrieved information is correlated to a plurality of historical information by applying weighting factors to normalize the retrieved information to the current CT image. By adjusting one or more variables or piece of information related to the patient targeted for surgery, the digital representation of the anatomical structure is created. The digital representation of the anatomical structure will react in a similar manner as the anatomical structure would react in real life allowing for simulations or other predictive models to be run on the anatomical structure representation to determine potential medical outcomes, unforeseen issues or other predictive outcomes after and during surgery.

In one embodiment, new images, measurements and readings are extracted from real time video of the surgical procedure. Consistent with this embodiment, the surgeon may be alerted to additional unforeseen issues during surgery that are predicted based on real time surgical information. In another embodiment, the information is fed into a robotic surgical device with the robotic surgical device modifying operational parameters based on predictive information from the pre-surgical analytic system 100.

As another illustrative example, the pre-surgical analytic system 100 may identify morphology or outliers for integration with robotic or extended reality assistance with surgery. As another illustrative example, the pre-surgical analytic system 100 may but is not limited to, identity 3D patient morphology to preoperatively plan a generalized approach such as mechanical alignment, kinematic alignment, implant sizing, and implant position.

FIG. 11 depicts a schematic representation of a process performed by the pre-surgery analytic unit 102. In step 1102, a plurality of CT scan images of an anatomical structure is gathered. Each image is gathered as a 512×512-pixel image representing a slice of the anatomical structure in the axial view. In one embodiment, the CT scan images are images of a knee. In another embodiment, the CT scan images an image of a hip. In step 1104, images of interest are identified from the plurality of CT scan images. Images are analyzed to determine the best images to gather measurements of the different aspects of the anatomical structure. In step 1106, each of the images of interest are converted to binary representations. In step 1108, measurements are taken from the binary representations of the images. In step 1110, a score related to a pre-surgical attribute is calculated based on the measurements.

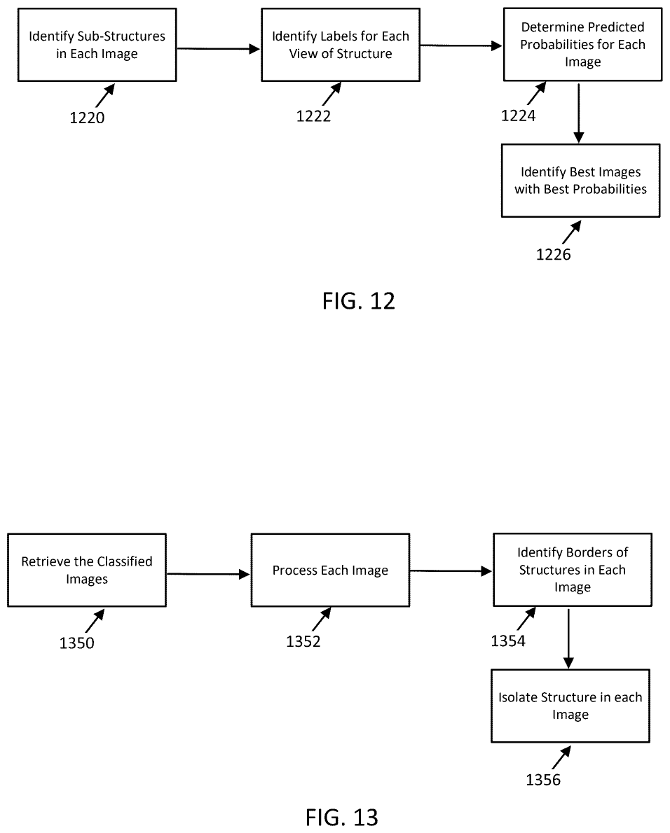

FIG. 12 depicts a schematic representation of a process performed by the pre-surgery analytic unit 102 to classify images. In step 1220, the information analysis unit 112 identifies substructures in each image. In one embodiment, a Keras library is used and loaded in convolutional layers of the VGG16 model with pretrained weights from the ImageNet dataset. The weights are frozen for the convolutional layers to avoid modification during training. Images are passed through the VGG16 model to obtain the feature vectors for each image. In step 1222, the information analysis unit 112 identifies labels in each structure. The labels represent different features of the anatomical structure. In one embodiment, each label is associated with a feature vector. In step 1224, the predicted probability of each image is determined. In one embodiment, the predicted probability is determined by passing the image vectors and labels through an XGBoost model that generates an array of predicted probabilities for each feature vector. In one embodiment, a linear scan of the array of probabilities is scanned to determine the image with the best probability. In step 1226, the images with the best probabilities are identified.

FIG. 13 depicts a schematic representation of a process performed by the pre-surgery analytic unit 102 to segment images. In step 1350, each classified image is retrieved and prepared for processing. In step 1352, each image is processed by a UNet. The image may be processed by an Attention UNet, Unet3+ and/or 2D Transformer UNet. In one embodiment, each image is passed through a single UNet. In another embodiment, each image is parallel processed by at least two UNets. In step 1354, the boundaries of the anatomic structures are identified in the processed images. In step 1356, the identified structures in each image are isolated from any background image or noise as a binary mask.

While various embodiments of the present invention have been described, it will be apparent to those of skill in the art that many more embodiments and implementations are possible that are within the scope of this invention. Accordingly, the present invention is not to be restricted except in light of the attached claims and their equivalents.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter. Further, the use of the terms “include,” “includes” and “including” means include, includes and or including as well as include, includes and including, but not limited to.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and does not constitute any admission as to the correctness of the dates or contents of these documents. Lastly, the terminology used herein is for the purpose of describing particular embodiments only and is not intended to limit the scope of the present invention, which is defined solely by the claims. Accordingly, the present invention is not limited to that precisely as shown and described.

What is claimed:

1. A pre-surgical analytical system including:
a plurality of images gathered from a CT scan;
a classification unit that classifies the identifies landmark images from the plurality of images;
a segmentation unit that segments each of the landmark image;
an image analysis unit that calculates a plurality of predetermined angles and measurements of anatomical structures in each image;
wherein,
the image analysis unit compares each measurement and angle to predetermined thresholds and determines the number of angles and measurements are outside the predetermined threshold; and
the image analysis unit weighs each of the angles and measurements outside the predetermined thresholds and calculates a score indicating a potential outcome of a surgical procedure.

2. The pre-surgical analytical system of claim 1, wherein the images are configured to a 1 mm×1 mm×1 mm voxel prior to analysis.

3. The pre-surgical analytical system of claim 1, wherein each image is a two-dimensional slice of an anatomical structure.

4. The pre-surgical analytical system of claim 1, wherein a deep learning computer model is applied to each image to identify each landmark image.

5. The pre-surgical analytical system of claim 4, wherein the deep learning computer model is a VGG16-XGBoost or similar classification model.

6. The pre-surgical analytical system of claim 1, wherein the anatomical structure is the knee.

7. The pre-surgical analytical system of claim 1, wherein the anatomical structure is the hip.

8. The pre-surgical analytical system of claim 1, wherein the threshold values are based on historical surgical information.

9. The pre-surgical analytical system of claim 1, wherein 27 unique angles and measurements are taken from each image.

10. The pre-surgical analytical system of claim 1, wherein the angles and measurements include the rotational measurements of the anatomical structure.

11. A method of performing a pre-surgical analysis using a pre-surgical analytical system including the steps of:
gathering a plurality of images from a CT scan;
classifying the identified landmark images from the plurality of images;
segmenting each of the landmark image;
calculating a plurality of predetermined angles and measurements of anatomical structures in each image;
comparing each measurement and angle to predetermined thresholds and determining the number of angles and measurements that are outside the predetermined threshold; and
weighing each of the angles and measurements outside the predetermined thresholds and calculating a score indicating a potential outcome of a surgical procedure.

12. The method of claim 11, including the step of configuring each image to a 1 mm×1 mm×1 mm voxel prior to analysis.

13. The method of claim 11, wherein each image is a two-dimensional slice of an anatomical structure.

14. The method of claim 11, including the step of applying a deep learning computer model to each image to identify each landmark image.

15. The method of claim 14, wherein the deep learning computer model is a VGG16-XGBoost or similar classification model.

16. The method of claim 11, wherein the anatomical structure is the knee.

17. The method of claim 11, wherein the anatomical structure is the hip.

18. The method of claim 11, wherein the threshold values are based on historical surgical information.

19. The method of claim 11, wherein 27 unique angles and measurements are taken from each image.

20. The method of claim 11, wherein the angles and measurements include the rotational measurements of the anatomical structure.
